(19) United States
(12) Patent Application Publication
(10) Pub. No.: US 2020/0282190 A1
(43) Pub. Date: Sep. 10, 2020

(54) DRUG TRANSFER DEVICE AND METHOD FOR FORMING DRUG LAYER

(71) Applicant: TERUMO KABUSHIKI KAISHA, Tokyo (JP)

(72) Inventors: Yasuo KUROSAKI, Kanagawa (JP); Hiroshi GOTO, Kanagawa (JP)

(73) Assignee: TERUMO KABUSHIKI KAISHA, Tokyo (JP)

(21) Appl. No.: 16/879,002

(22) Filed: May 20, 2020

Related U.S. Application Data
(63) Continuation of application No. PCT/JP2018/043179, filed on Nov. 22, 2018.

(30) Foreign Application Priority Data
Nov. 22, 2017 (JP) 2017-224337

(51) Int. Cl. A61M 25/10; B05D 1/28

(52) U.S. Cl. CPC A61M 25/1029; A61M 2025/1031; B05D 1/286

(57) ABSTRACT

A drug transfer device and a method for forming a drug layer are disclosed, which can quickly and easily provide an appropriate amount of a drug on a surface of a medical instrument. The drug transfer device is configured to transfer a drug to a surface of a balloon, the drug transfer device including a heat-shrinkable tube, and a drug layer provided on an inner surface of the heat-shrinkable tube.

### CROSS-REFERENCES TO RELATED APPLICATIONS

This application is a continuation of International Application No. PCT/JP2018/043179 filed on Nov. 22, 2018, which claims priority to Japanese Application No. 2017-224337 filed on Nov. 22, 2017, the entire content of both of which is incorporated herein by reference.

### FIELD OF THE DISCLOSURE

The present disclosure generally relates to a drug transfer device configured to transfer a drug to a surface of a medical instrument such as a balloon, and a method for forming a drug layer on a surface of a medical instrument.

### BACKGROUND DISCUSSION

In recent years, a balloon catheter has been used to improve a lesion (stenotic part) generated in a body lumen. The balloon catheter typically includes an elongated shaft portion and a radially inflatable balloon provided on a distal side of the shaft portion. When the deflated balloon is inflated after reaching a target location in a body via a relatively thin body lumen, a lesion can be pushed to be widened.

However, when the lesion is forcibly pushed to be widened, smooth muscle cells may excessively proliferate to cause new stenosis (restenosis) at the lesion. Therefore, recently, a drug eluting balloon (DEB) in which a surface of the balloon is coated with a drug for suppressing stenosis has been used. The drug eluting balloon inflates to instantaneously release the drug with which the surface has been coated to the lesion, thereby suppressing restenosis.

As a method for forming a drug layer on a surface of a balloon, for example, U.S. Pat. No. 8,597,720 B2 describes a method of spraying a solution containing a drug on a balloon, a dipping method, a coating method using a brush, a coating method using a rotating body, and a method of supplying a solution using a pipette.

Japanese Patent Application Publication No. 2007-510446 A describes a method in which a coating material containing a drug is attached to a printing roller, and then the coating material is transferred from the printing roller to adhere on an implant.

With the methods described in U.S. Pat. No. 8,597,720 B2 and Japanese Patent Application Publication No. 2007-510446 A, it is difficult to quickly and easily provide an appropriate amount of a drug on a surface of a balloon.

### SUMMARY

A drug transfer device and a method for forming a drug layer are disclosed which can quickly and rather easily provide or apply an appropriate amount of a drug on a surface of a medical instrument.

A drug transfer device is disclosed that is configured to transfer a drug to a surface of a medical instrument used by being inserted into a living body, and includes: a heat-shrinkable tube; and a drug layer provided on an inner surface of the heat-shrinkable tube.

A method is disclosed for forming a drug layer which provides a drug on a surface of a medical instrument used by being inserted into a living body, and includes: covering the medical instrument with a drug transfer device in which an inner surface of a heat-shrinkable tube is coated with a drug layer; transferring the drug layer to the surface of the medical instrument by heating the heat-shrinkable tube to shrink; and removing the heat-shrinkable tube from the drug layer.

A method is disclosed for forming a drug layer which provides a drug on a surface of a balloon configured to be inserted into a living body, the method comprising: injecting a predetermined amount of an inflation fluid into the balloon to inflate the balloon; inserting the inflated balloon into a through-hole of a heat-shrinkable tube, the heat-shrinkable tube having an inner surface coated with a drug layer; transferring the drug layer to the surface of the inflated balloon by heating the heat-shrinkable tube; and removing the heat-shrinkable tube from the drug layer on the surface of the balloon.

The heat-shrinkable tube shrinks when the drug transfer device configured as described above is heated in the state of covering the medical instrument. As a result, the drug layer is transferred to the surface of the medical instrument by a shrinking force of the heat-shrinkable tube, and an appropriate amount of the drug can be relatively quickly and rather easily provided on the surface of the medical instrument.

The drug transfer device may further include an adhesive layer provided on an inner surface of the drug layer. As a result, the adhesive layer can adhere to the surface of the medical instrument, and the drug layer can be effectively provided on the surface of the medical instrument.

The drug layer may be water-insoluble and the adhesive layer may be water-soluble. As a result, when the water-soluble adhesive layer is formed on the inner surface of the water-insoluble drug layer, the dissolution of the drug in the adhesive layer can be suppressed by a material of the adhesive layer containing water. In addition, the medical instrument contains moisture at the time of transferring the drug layer, favorable adhesiveness is exhibited, and the dissolution of the drug can be suppressed.

The drug in the drug layer may contain at least one selected from the group including rapamycin, paclitaxel, docetaxel, and everolimus. As a result, restenosis of a stenotic part in a blood vessel can be favorably suppressed by the drug layer.

The drug in the drug layer may contain at least one selected from the group including a water-insoluble drug, a water-soluble drug, and a hydrophilic polymer. As a result, it is possible to apply, to the drug layer, a drug that is appropriate for conditions and the like, alone or in combination, from various drugs.

The adhesive layer may exhibit an adhesive force when heated. As a result, the adhesive layer can also be heated when the heat-shrinkable tube is heated, and the adhesive layer can exhibit the adhesive force. Therefore, it is possible to prevent the adhesive layer from adhering to an unintended position before the heating. Therefore, the heat-shrinkable tube can be attached to an appropriate position on the surface of the medical instrument after positioning the drug layer with respect to the medical instrument with relatively high precision.

The heat-shrinkable tube may have a fragile portion that is more fragile than other portions of the heat-shrinkable tube. As a result, the heat-shrinkable tube that has been heat-shrunk can be rather easily removed from the surface of the medical instrument by breaking the fragile portion.

The medical instrument may be a balloon capable of inflating and deflating. As a result, the drug layer can be relatively quickly and rather easily provided on a surface of the balloon.

In the method for forming a drug layer configured as described above, the medical instrument is covered with the drug transfer device and heated, and thus, the drug layer can be transferred to the surface of the medical instrument by the shrinking heat-shrinkable tube. Therefore, the medical instrument coated with the drug can be rather easily formed.

In the transferring of the drug layer to the surface of the medical instrument, an adhesive layer provided on an inner surface of the drug layer may be attached to the surface of the medical instrument. As a result, the adhesive layer can be attached to the surface of the medical instrument, and the drug layer can be effectively provided on the surface of the medical instrument.

In the covering of the medical instrument with the drug transfer device, the medical instrument removed from an inside of the living body is covered with the drug transfer device. As a result, the medical instrument that has been used in the living body can be removed from the living body, and then, the drug layer can be provided on the same medical instrument for reuse.

The medical instrument may be a balloon capable of inflating and deflating, guidewire, guiding sheath, guiding catheter, or stent. As a result, it is possible to relatively quickly and rather easily provide an appropriate amount of the drug layer on a surface of the balloon, the guidewire, the guiding sheath, the guiding catheter, or the stent. If the medical instrument is the balloon, the appropriate amount of the drug layer can be relatively quickly and rather easily provided on the surface of the balloon. In addition, the balloon that has been used for pre-dilation of a target site of the living body can be removed, and then, the drug layer can be provided on the same balloon to reuse the balloon for post-dilation of the target site.

### BRIEF DESCRIPTION OF THE DRAWINGS

FIG. 1 is a perspective view illustrating a drug transfer device according to an embodiment disclosed here.

FIG. 2 is a cross-sectional view taken along line II-II of FIG. 1;

FIG. 3 is a front view illustrating a balloon catheter.

FIG. 4 is a front view illustrating a distal portion of the balloon catheter.

FIG. 5 is a cross-sectional view taken along line V-V of FIG. 4;

FIG. 6 is a plan view illustrating a state where the drug transfer device covers a balloon.

FIG. 7 is a cross-sectional view taken along line VII-VII of FIG. 6.

FIG. 8 is a cross-sectional view illustrating a state where the drug transfer device is contracted.

FIG. 9 is a plan view illustrating a state where a fragile portion of the drug transfer device is broken.

FIG. 10 is a cross-sectional view taken along line X-X of FIG. 9 illustrating a state where a heat-shrinkable tube is removed from the balloon.

FIGS. 11A and 11B are cross-sectional views illustrating a modification of a drug layer transferred to the balloon, FIG. 11A illustrates a state where a drug transfer device is further provided outside the drug layer transferred to an outer surface of the balloon, and FIG. 11B illustrates a state where a drug layer is further transferred to the outside of the drug layer transferred to the outer surface of the balloon.

FIGS. 12A and 12B are plan views illustrating another modification of the drug layer transferred to the balloon, FIG. 12A illustrates a state where the drug layers are provided side by side, and FIG. 12B illustrates a state where the drug layers are provided side by side to overlap each other.

FIG. 13 is a plan view illustrating still another modification of the drug layer of the drug transfer device.

### DETAILED DESCRIPTION

Set forth below with reference to the accompanying drawings is a detailed description of embodiments of a drug transfer device configured to transfer a drug to a surface of a medical instrument such as a balloon, and a method for forming a drug layer on a surface of a medical instrument representing examples of the inventive drug transfer device configured to transfer a drug to a surface of a medical instrument such as a balloon and method for forming a drug layer on a surface of a medical instrument disclosed here. Note that dimensional ratios of the drawings are exaggerated for the convenience of description and may differ from actual ratios in some cases.

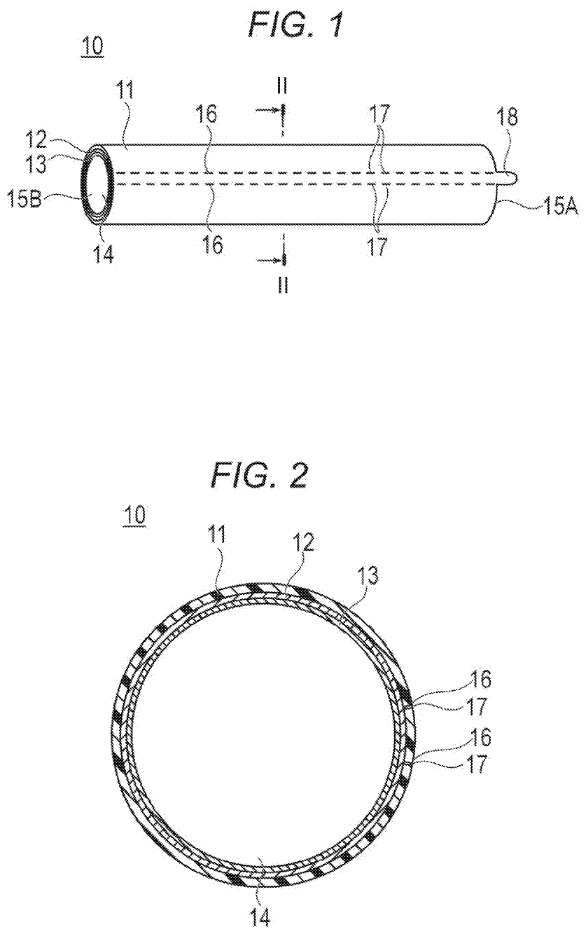

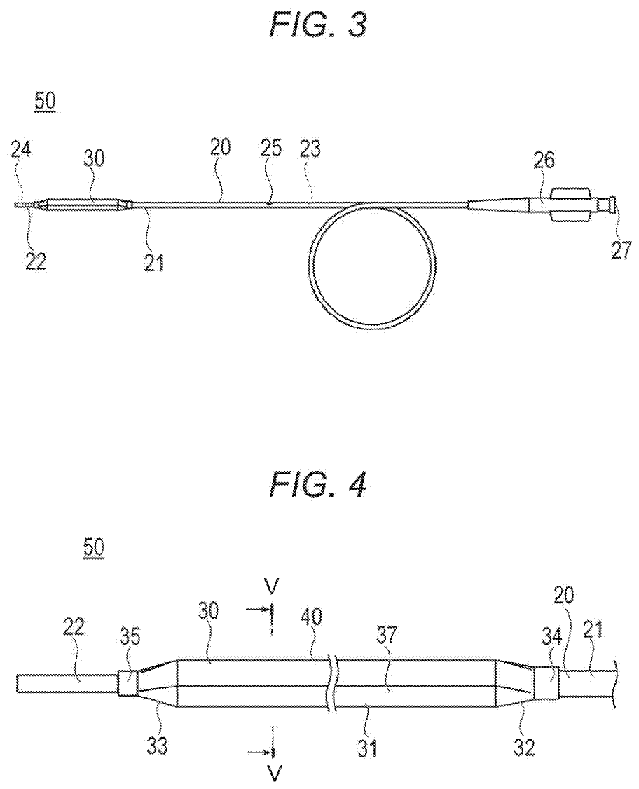

A drug transfer device 10 according to the embodiment of the present disclosure is a device configured to provide a drug layer on a surface of a balloon 30 (see FIG. 3), which is inserted into a stenotic part of a living body lumen, such as a blood vessel, to push and widen the stenotic part, thereby forming a drug-eluting balloon as illustrated in FIGS. 1 and 2. Note that a medical instrument on which the drug transfer device 10 provides the drug layer is not limited to the balloon 30, and may be, for example, a guide wire, a guiding sheath, a guiding catheter, a stent, or the like. Hereinafter, a case where the drug layer is provided on the balloon 30 by the drug transfer device 10 will be described.

The drug transfer device 10 can include: a heat-shrinkable tube 11 that shrinks when heated; a drug layer 12 containing a drug; and an adhesive layer 13 having an adhesive force.

In accordance with an embodiment, the heat-shrinkable tube 11 is a tube (i.e., tubular member) having a diameter that is reduced when heated. The heat-shrinkable tube 11 has such a strength that the heat-shrinkable tube 11 can maintain a through-hole 14. Note that a cylindrical film may be used. The cylindrical film can be flexible and relatively thin, and thus, does not always have such strength as to be capable of maintaining the through-hole 14 and can be deformed into a flat plate shape such that the through-hole 14 is closed.

The material of the heat-shrinkable tube 11 is not limited as long as the material of the heat-shrinkable tube 11 can be reduced in diameter by heating. However, the heat-shrinkable tube 11 is preferably made of a material capable of coating an inner circumferential surface of the heat-shrinkable tube with a drug. The heat-shrinkable tube 11 preferably shrinks at a relatively low heating temperature. The temperature at which the heat-shrinkable tube 11 shrinks can be, for example, 40° C. to 150° C., preferably 40° C. to 100° C. Since the heat-shrinkable tube 11 shrinks at the relatively low temperature, deterioration of the drug, deformation of the balloon 30, and the like can be relatively suppressed. A shrinkage ratio of an inner diameter of the heat-shrinkable tube 11 (inner diameter after shrinkage/inner diameter before shrinkage) is not particularly limited, for example, 40% to 80%.

A thickness of the heat-shrinkable tube 11 is not particularly limited, but is, for example, 0.01 mm to 0.20 mm, and preferably 0.03 mm to 0.08 mm.

The material of the tubular heat-shrinkable tube 11 can include, for example, polyolefin, a fluorine-based polymer, polyvinyl chloride, silicone, thermoplastic elastomer, or the like. A thickness of the tubular heat-shrinkable tube 11 is not particularly limited, but can be, for example, 0.01 mm to 0.2 mm, and preferably 0.03 mm to 0.08 mm.

The material of the heat-shrinkable tube 11 in the case of a tubular film can include, for example, polyolefin, polyvinyl chloride, polystyrene, polyethylene, polypropylene, polyethylene terephthalate, or the like. A thickness of the heat-shrinkable tube 11, which is a tubular film, is not particularly limited, but can be, for example, 0.005 mm to 0.25 mm, and preferably 0.01 mm to 0.10 mm. A shrinkage ratio of an inner diameter of the heat-shrinkable tube 11, which is a film, is not particularly limited, but can be, for example, 10% to 80%.

In accordance with an embodiment, the heat-shrinkable tube 11 can include two fragile portions 16 extending from one opening 15A to the other opening 15B of the through-hole 14. The two fragile portions 16 can be arranged in parallel and configured to be separated at an angle of 180 degrees or smaller in the circumferential direction of the heat-shrinkable tube 11. Note that the two fragile portions 16 are not necessarily parallel. The two fragile portions 16 can be perforations formed by arranging, for example, a plurality of small holes 17 side by side. Note that the fragile portion 16 may be a groove extending from the one opening (i.e., an open end) 15A to the other opening (i.e., an other open end) 15B. Alternatively, the fragile portion 16 may be a slit. The heat-shrinkable tube 11 can include a grip portion 18 sandwiched between the two fragile portions 16 at an edge of the one opening 15A. The grip portion 18 is a portion to be gripped by a finger, and protrudes from the edge of the opening 15A so as to be easily gripped.

In accordance with an embodiment, the drug layer 12 is provided on an inner circumferential surface of the heat-shrinkable tube 11. Note that it is preferable that the drug layer 12 is not provided on the grip portion 18 so as not to adhere to the finger. The drug contained in the drug layer 12 may be water-soluble or water-insoluble drug. A water-insoluble drug means a drug that is insoluble or poorly soluble in water, and specifically solubility in water may be less than 1 mg/mL, and further, may be less than 0.1 mg/mL. Water-insoluble drugs include fat-soluble drugs. An amount of the drug contained in the drug layer 12 is not particularly limited, but the drug can be contained, for example, at a density of 0.1 μg/mm² to 10 μg/mm², preferably at a density of 0.5 μg/mm² to 5 μg/mm², and more preferably at a density of 0.5 μg/mm² to 3.5 μg/mm². A thickness of the drug layer 12 is not particularly limited, but can be, for example, 0.1 μm to 100 μm, preferably 0.5 μm to 50 μm, and more preferably 0.5 μm to 10 μm or 10 μm to 30 μm. A form of the water-insoluble or water-soluble drug is not particularly limited, and may be, for example, a crystal or not.

The water-insoluble drug can include, for example, immunosuppressants, for example, cyclosporines containing cyclosporine, immunoadjuvants such as rapamycin, carcinostatics such as paclitaxel, antiviral agents or antibacterial agents, antineoplastic agents, analgesic agents and anti-inflammatory agents, antibiotics, antiepileptics, anxiolytic agents, antiparalytic agents, antagonists, neuron blocking agents, anticholinergic agents and cholinergic agents, muscarine antagonists agents and muscarine agents, antiadrenergic agents, antiarrhythmic agents, antihypertensive agents, hormone preparations, and nutritional supplements.

The water-insoluble drug is preferably, for example, at least one selected from the group including rapamycin, paclitaxel, docetaxel, and everolimus. The rapamycin, paclitaxel, docetaxel, and everolimus in the present disclosure can include their analogs and/or derivatives as long as the analogs and/or derivatives have equivalent drug effect. For example, paclitaxel and docetaxel are in an analog relation. Rapamycin and everolimus are in a derivative relation among these, paclitaxel is more preferable.

The water-soluble drug may be a drug having solubility in water of 1 mg/mL or more, preferably 5 mg/mL or more, more preferably 10 mg/mL or more, and still more preferably 33 mg/mL or more. Water-soluble antiplatelet drugs can include, for example, clopidogrel sulfate, ticlopidine hydrochloride, prasugrel hydrochloride, sarpogrelate hydrochloride, and the like (incidentally, water-insoluble antiplatelet drugs include aspirin, cilostazol, ticagrelor, and the like). Examples of the water-soluble anticoagulant can include warfarin, edoxaban tosilate hydrate, heparin, dabigatran etexilate methanesulfonate, and the like. The drug may also be a hydrophilic polymer, and a wet coating using the hydrophilic polymer (the coating that exhibits lubricity when wetted with water) is possible. The drug may be applied as the hydrophilic polymer on a surface (inner and outer surfaces) of a medical instrument to be inserted into a blood vessel (for example, a guidewire, a guiding sheath, a guiding catheter, or the like) without being limited to the surface (inner and outer surfaces) of the balloon catheter.

The drug layer 12 may contain an additive (for example, an excipient). When the drug layer 12 contains the additive, the additive can be, for example, a water-soluble low molecular weight compound or biodegradable polymer. A molecular weight of the water-soluble low molecular weight compound is 50 to 2,000, preferably 50 to 1,000, more preferably 50 to 500, and still more preferably 50 to 200. An amount of the water-soluble low molecular weight compound is preferably 10 parts by weight to 5,000 parts by weight, more preferably 50 parts by weight to 3000 parts by weight, and still more preferably 100 parts by weight to 1000 parts by weight, per 100 parts by weight of the water-insoluble drug. The water-soluble low molecular weight compound material can be, for example, an amino acid ester such as a serine ethyl ester, a saccharide such as glucose, a sugar alcohol such as sorbitol, a citrate ester, polysorbate, glycerin, propylene glycol, polyethylene glycol, polyglycerin, urea, a water-soluble polymer, a contrast agent, a glycerol ester of a short-chain monocarboxylic acid, pharmaceutically acceptable salts and surfactants, and the like, or a mixture of two or more of the water-soluble low molecular weight compound materials disclosed herein can be used. The water-soluble low molecular weight compound has a hydrophilic group and a hydrophobic group, and can be characterized by being soluble in water. The water-soluble low molecular weight compound is preferably non-swellable or hardly swellable (i.e., only a part of the compound is swellable). The additive containing the water-soluble low molecular weight compound has an effect of uniformly dispersing the water-insoluble drug on the surface of the heat-shrinkable tube 11. In accordance with an embodiment, it can be preferable that the additive is not a hydrogel. The additive contains the low molecular weight compound, and thus, dissolves relatively quickly without swelling when coming into contact with an aqueous solution. Further, the additive can rather easily dissolve when the balloon 30 is inflated in the blood vessel so that crystal particles of the water-insoluble drug on the surface of the balloon 30 can be easily released, and thus, there is an effect of increasing the number of the crystal particles of the drug adhering to the blood vessel. Examples of the biodegradable polymer can include polyglycolic acids, polylactic acids, poly (lactide-co-glycolide) copolymers, polydioxanone, polycaprolactone, polyethylene glycol-polyester diblock copolymers, and the like. A molecular weight of the biodegradable polymer is 4,000 to 25,000, preferably 4,000 to 100,000, and more preferably 4,000 to 50,000.

The water-soluble low molecular weight compound has a molecular weight of 50 to 2,000, and can be dissolved at an amount of 1 mg/mL or more in water, preferably dissolved at an amount of 5 mg/mL or more in water, more preferably dissolved at an amount of 10 mg/mL or more in water, still more preferably dissolved at an amount of 33 mg/mL or more in water, and preferably dissolved in water without inflating. In accordance with an exemplary embodiment, it can be preferable that the water-soluble low molecular weight compound is not a hydrogel. The water-soluble low molecular weight compound is preferably not a polymer, and more preferably not a water-insoluble polymer. In accordance with an exemplary embodiment, it can be preferable that the water-soluble low molecular weight compound is not polyethylene glycol (PEG) and a water-soluble PEG (for example, polyethylene glycol 200-600).

The solubility of a substance can be defined as a degree of dissolution within 30 minutes at 20° C. For example, the solubility of a substance can be defined by an amount of solvent (for example, an amount of water) required to dissolve 1 g (or 1 mL) of solute. When the amount of solvent required to dissolve 1 g of solute is less than 1 mL, the solute is extremely soluble in the solvent. In cases of extremely soluble, the amount of dissolved solute is more than 1000 mg/mL. Examples of extremely soluble substances can include sorbitol, urea, and glycerol. When the amount of solvent required to dissolve 1 g of solute is 1 mL or more and less than 10 mL, the solute is freely soluble in the solvent. In cases of freely soluble, the amount of dissolved solute may be more than 100 mg/mL and 1000 mg/mL or less. Examples of freely soluble substances can include polysorbate, an amino acid ester, polyethylene glycol 200-600, a serine ethyl ester, a contrast agent (iopromide), and a water-soluble polymer. When the amount of solvent required to dissolve 1 g of solute is 10 mL or more and less than 30 mL, the solute is soluble in the solvent. In cases, of soluble solvents, the amount of dissolved solute may be more than 33 mg/mL and 100 mg/mL or less. Examples of soluble solvents can include polyethylene glycol. When the amount of solvent required to dissolve 1 g of solute is 30 mL or more and less than 100 mL, the solute is slightly soluble in the solvent. In cases of slightly soluble, the amount of dissolved solute may be more than 10 mg/mL and 33 mg/mL or less. When the amount of solvent required to dissolve 1 g of solute is 100 mL or more and less than 1000 mL, the solute is sparingly soluble in the solvent. In cases of sparingly soluble, the amount of dissolved solute may be more than 1 mg/mL and 10 mg/mL or less. When the amount of solvent required to dissolve 1 g of solute is 1000 mL or more and less than 10,000 mL, the solute is extremely insoluble in the solvent. In cases of extremely insoluble, the amount of dissolved solute may be more than 0.1 mg/mL and 1 mg/mL or less. When the amount of solvent required to dissolve 1 g of solute is 10,000 mL or more, the solute is hardly soluble in the solvent. In cases of hardly soluble, the amount of dissolved solute may be0.1 mg/mL or less. Examples of hardly soluble substances can include a fatty acid ester of glycerin. The water-soluble substance refers to a substance other than a substance that is “extremely insoluble” and a substance that is “hardly soluble”. Specifically, the water-soluble substance indicates a substance that is “extremely soluble”, a substance that is “freely soluble”, a substance that is “slightly soluble”, and a substance that is “sparingly soluble”. The water-soluble substance preferably indicates a substance that is “extremely soluble”, a substance that is “freely soluble” and a substance that is “slightly soluble”.

In accordance with an exemplary embodiment, the adhesive layer 13 is provided on an inner circumferential surface of the drug layer 12. The adhesive layer 13 is a layer adhering to the surface of the balloon 30. A thickness of the adhesive layer 13 is not limited, for example, the thickness of the adhesive layer 13 can be 0.01 μm to 50 μm, preferably 0.1 μm to 30 μm, and more preferably 0.1 μm to 5 μm.

The material of the adhesive layer 13 is preferably water-soluble, but is not limited to materials that are water-soluble. Examples of the water-soluble material forming the adhesive layer 13 can include a poly (lactide-co-glycotide) copolymer, a polymer such as polycaprolactone, a surfactant such as polyethylene glycol, a polyoxyethylene fatty acid diester, and a polyoxyethylene fatty acid monoester, a polyoxyethylene polyoxypropylene block polymer, polysorbate 20, polysorbate 80, and polyoxyethylene hydrogenated castor oil, a solvent such as glycerin and propylene glycol, gelatin-resorcin formaldehyde, an α-cyanoacrylate-based adhesive, and a fibrin-based adhesive used as medical adhesives, and the like. In addition, the material forming the adhesive layer 13 may be, for example, a material that exhibits an adhesive force by raising its temperature to a temperature at which the heat-shrinkable tube 11 is heated. Examples of the material that exhibits the adhesive force when heated can include a poly (lactide-co-glycotide) copolymer, a polymer such as polycaprolactone, a surfactant such as polyethylene glycol, a polyoxyethylene fatty acid diester, a polyoxyethylene fatty acid monoester, and a polyoxyethylene polyoxypropylene block polymer, an α-cyanoacrylate adhesive, and a fibrin adhesive used as medical adhesives, and the like.

The drug transfer device 10 is used with the balloon 30 housed in the drug transfer device 10. Therefore, an inner diameter of the drug transfer device 10 is preferably larger than an outer diameter of the balloon 30. The drug transfer device 10 can provide the drug layer on the surface of the inflated balloon 30 or the deflated balloon 30. Therefore, the inner diameter of the drug transfer device 10 is preferably larger than an outer diameter of the inflated balloon 30 when the drug transfer device 10 provides the drug layer on the surface of the inflated balloon 30. The inner diameter of the drug transfer device 10 is preferably larger than an outer diameter of the deflated balloon 30 when the drug transfer device 10 provides the drug layer on the surface of the deflated balloon 30. The inner diameter of the drug transfer device 10 can be, for example, 1.5 mm to 20 mm, preferably 1.5 mm to 15 mm, and more preferably 1.5 mm to 10 mm.

A length from the opening 15A to the opening 15B of the drug transfer device 10 is preferably equal to or longer than an axial length of the balloon 30 in a range where the drug is arranged. The length of the drug transfer device 10 can be, for example, 10 mm to 400 mm, preferably 15 mm to 350 mm, and more preferably 20 mm to 300 mm.

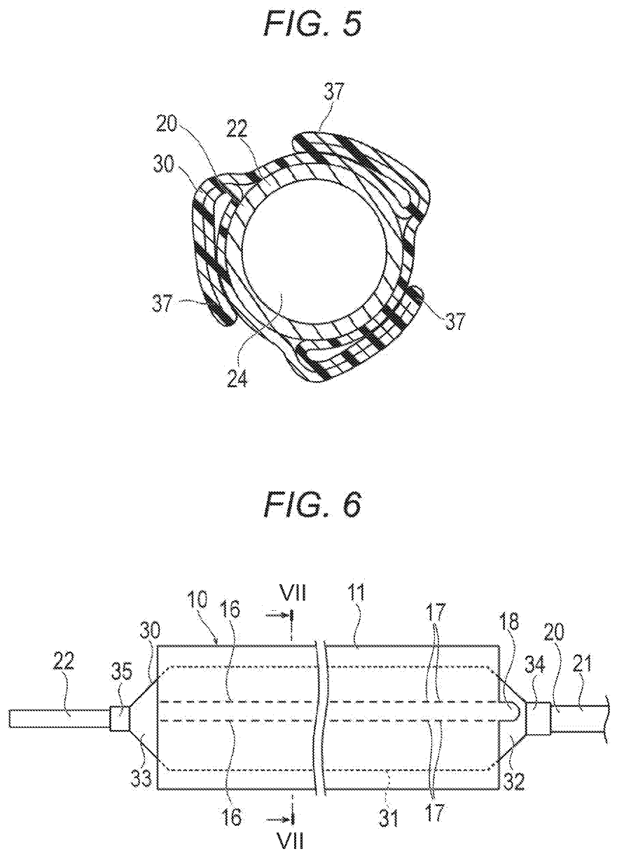

Next, a balloon catheter 50 on which a drug is provided using the drug transfer device 10 will be described with reference to FIGS. 3 to 5. In the present disclosure, a side of the balloon catheter 50 to be inserted into a living body lumen is referred to as a “distal side” and an operating hand side is referred to as a “proximal side”.

The balloon catheter 50 can include an elongated shaft portion 20, the balloon 30 provided at a distal portion of the shaft portion 20, and a hub 26 fixed to a proximal end of the shaft portion 20.

The shaft portion 20 can include an outer tube 21 that is a tube body of which distal end and proximal end are open and an inner tube 22 which is a tubular body provided inside the outer tube 21. The inner tube 22 is housed in a hollow inside of the outer tube 21, and the shaft portion 20 has a double-tube structure at the distal portion. The hollow inside of the inner tube 22 can be a guide wire lumen 24 through which a guide wire is inserted. An inflation lumen 23 for circulating inflation fluid of the balloon 30 is formed in the hollow inside of the outer tube 21 outside the inner tube 22. The inner tube 22 is open to the outside at a side opening 25. The inner tube 22 can protrude further to the distal side from the distal end of the outer tube 21. A distal tip, which is a separate member, may be provided at a distal portion of the inner tube 22.

The balloon 30 (medical instrument) can include a straight portion 31 formed at the center in the axial direction, a proximal tapered portion 32 located on the proximal side of the straight portion 31, and a distal tapered portion 33 located on the distal side of the straight portion 31. The straight portion 31 can have a cylindrical shape that has substantially the same outer diameter when inflated. An outer diameter of the proximal tapered portion 32 gradually decreases from the straight portion 31 toward the proximal side. An outer diameter of the distal tapered portion 33 gradually decreases from the straight portion 31 toward the distal side.

In accordance with an exemplary embodiment, the straight portion 31 is a portion where the drug is provided by the drug transfer device 10. Note that the range in which the drug is provided by the drug transfer device 10 is not limited only to the straight portion 31, but may include at least a part of the proximal tapered portion 32 and the distal tapered portion 33 in addition to the straight portion 31. Alternatively, the range in which the drug is provided by the drug transfer device 10 may be only a part (or portion) of the straight portion 31.

In the balloon 30, a balloon fusing portion 34 located at the proximal end of the proximal tapered portion 32 can be fused at the distal portion of the outer tube 21. In addition, a balloon fusing portion 35 located at the distal end of the distal tapered portion 33 can be fused to the distal portion of the inner tube 22 in the balloon 30. Note that a method for fixing the balloon 30 to the outer tube 21 and the inner tube 22 is not limited to the fusion, but may be, for example, adhesion. As a result, the inside of the balloon 30 communicates with the inflation lumen 23. The balloon 30 can be inflated by injecting the inflation fluid into the balloon 30 via the inflation lumen 23. In accordance with an embodiment, the inflation fluid may be a gas or a liquid, and, for example, a gas such as a helium gas, a CO₂ gas, an O₂ gas, an N₂ gas, an Ar gas, air, and a mixed gas, or a liquid such as a saline solution and a contrast agent can be used.

In accordance with an embodiment, the balloon 30 can have a plurality of blade portions 37 shaped to protrude in the radial direction. The blade portions 37 can be folded in the circumferential direction. The blade portion 37 is formed by a fold extending substantially in the axial direction of the balloon 30. In accordance with an embodiment, a length of the blade portion 37 in the long-axis direction (i.e., axial direction) does not exceed a length of the balloon 30. The number of the blade portions 37 is not particularly limited, and can be one to seven, for example, however, as shown in the present embodiment, the number of blade portions 37 is three. The plurality of blade portions 37 are preferably provided to be uniform in the circumferential direction of the balloon 30, but are not limited to being provided uniform in circumferential direction of the balloon 30.

The length of the balloon 30 in the axial direction is not particularly limited, but is preferably 5 mm to 500 mm, more preferably 10 mm to 300 mm, and still more preferably 20 mm to 200 mm. The outer diameter of the balloon 30 when inflated is not particularly limited, but can be, for example, 1 mm to 10 mm, and more preferably 1.5 mm to 8 mm.

It is preferable that the balloon 30 have a certain degree of flexibility and a certain degree of hardness such that the balloon 30 can be inflated when reaching a blood vessel, a tissue, or the like, and release the drug on the surface of the balloon 30. Specifically, the balloon 30 is made of metal or resin, but it is preferable that at least the surface of the balloon 30 on which the drug is provided be made of resin. The material of at least the surface of the balloon 30, for example, can be polyolefins such as polyethylene, polypropylene, polybutene, an ethylene-propylene copolymer, an ethylene-vinyl acetate copolymer, and an ionomer, or a mixture of two or more of the polyolefins, thermoplastic resins such as soft polyvinyl chloride resin, polyamide, a polyamide elastomer, a nylon elastomer, polyester, a polyester elastomer, polyurethane, and a fluororesin, a silicone rubber, a latex rubber, and the like can be used. In accordance with an exemplary embodiment, the material of at least the surface of the balloon 30, polyamides are preferably used.

In the hub 26, a proximal opening 27 is formed, which communicates with the inflation lumen 23 of the outer tube 21 and functions as a port for inflow and outflow of the inflation fluid.

Next, a method for manufacturing the drug transfer device 10 will be described.

First, a coating solution containing the drug is prepared. The coating solution is a solution or suspension containing the drug, and can include a water-insoluble drug, an organic solvent, and water. Note that the coating solution may contain an additive (excipient). In addition, the coating solution does not necessarily include water.

The organic solvent is not particularly limited, and can be, for example, tetrahydrofuran, acetone, glycerin, ethanol, methanol, dichloromethane, hexane, ethyl acetate, and the like.

Next, the coating solution containing the drug is injected or sprayed into the heat-shrinkable tube 11, and the heat-shrinkable tube 11 is rotated about the axis. In accordance with an exemplary embodiment, the coating solution spreads uniformly on the inner surface of the heat-shrinkable tube 11 as the heat-shrinkable tube 11 rotates. Thereafter, the organic solvent volatilizes so that the drug layer 12 containing at least one of a crystal and an amorphous substance of the drug is formed on the inner surface of the heat-shrinkable tube 11.

Next, an adhesive solution obtained by dissolving a material of the adhesive layer 13 in water is injected or sprayed into the heat-shrinkable tube 11, and the heat-shrinkable tube 11 is rotated about the axis. The adhesive solution can be spread uniformly on the inner surface of the drug layer 12 as the heat-shrinkable tube 11 rotates. Thereafter, the adhesive layer 13 is formed on the inner surface of the drug layer 12 as the water evaporates. As a result, the drug transfer device 10 illustrated in FIGS. 1 and 2 is completed. Note that the adhesive solution may contain an amount of another solvent (for example, ethanol) that is less than water. Since the adhesive solution does not contain an organic solvent or contains only a small amount of the organic solvent, the drug layer 12 containing the water-insoluble drug is not dissolved in the solvent of the adhesive solution.

Next, an operation of the drug transfer device 10 according to the present embodiment will be described.

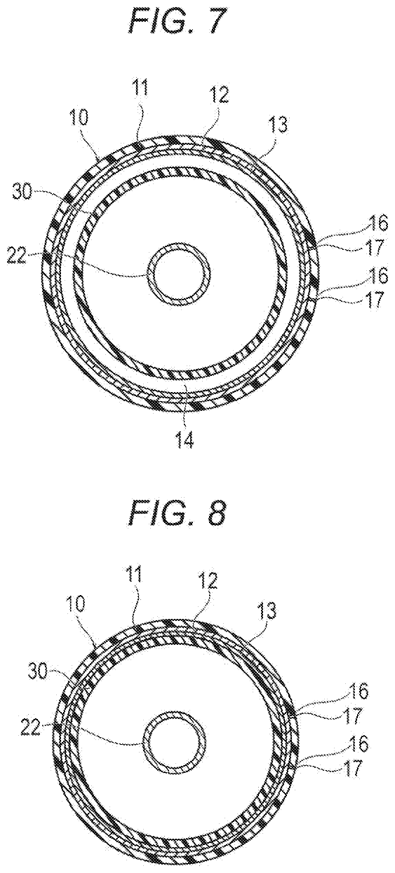

When arranging the drug on the balloon 30 by the drug transfer device 10, a predetermined amount of the inflation fluid is injected from the proximal opening 27 of the hub 26 using an indeflator (i.e., inflation/deflation device), a syringe, or the like to send the inflation fluid inside the balloon 30 through the inflation lumen 23 as illustrated in FIGS. 6 and 7. As a result, the folded balloon 30 inflates. Next, the balloon 30 is inserted into the through-hole 14 of the drug transfer device 10. Note that the balloon 30 may be inflated after the balloon 30 is inserted into the through-hole 14 of the drug transfer device 10. Alternatively, the balloon 30 inserted into the through-hole 14 of the drug transfer device 10 is not necessarily inflated. In this case, the drug is provided on the balloon 30 in a deflated state.

Next, the drug transfer device 10 is heated to a temperature at which the heat-shrinkable tube 11 shrinks by a dryer, an oven, or the like that supplies hot air when a current flows. As a result, the heat-shrinkable tube 11 is reduced in diameter, and the adhesive layer 13 is in contact with the balloon 30 as illustrated in FIG. 8. As a result, the adhesive layer 13 adheres to the surface of the balloon 30.

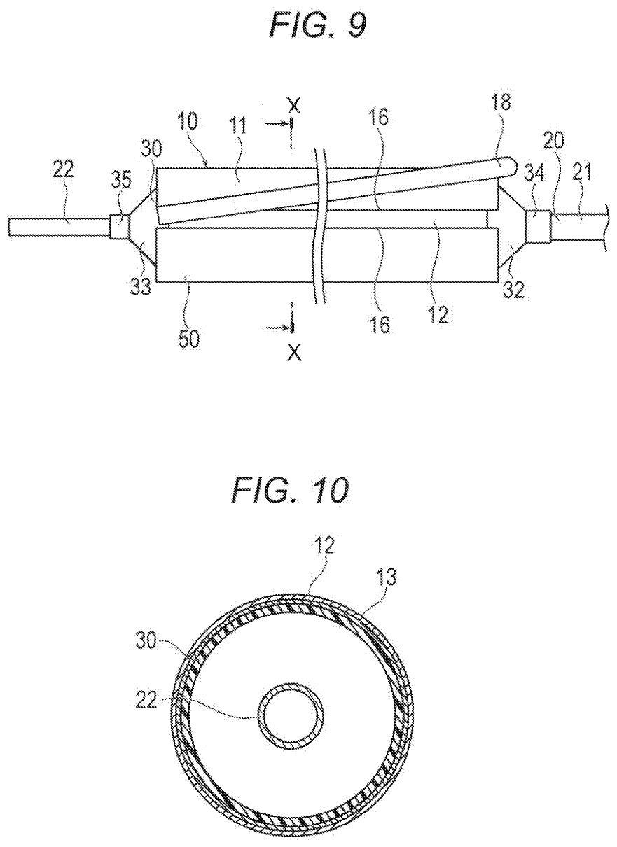

Next, the grip portion 18 is gripped and pulled outward in the radial direction of the balloon 30 as illustrated in FIG. 9. As a result, the heat-shrinkable tube 11 is broken at the fragile portion 16. Next, the heat-shrinkable tube 11 is peeled off from the balloon 30. At this time, it can be relatively easy to peel off the heat-shrinkable tube 11 from the balloon 30 since the heat-shrinkable tube 11 is broken at the fragile portion 16. The drug layer 12 is attached to the balloon 30 by the adhesive layer 13 as illustrated in FIG. 10. Therefore, the drug layer 12 is separated from the heat-shrinkable tube 11 and remains in the state of adhering to the surface of the balloon 30. As a result, the drug layer 12 is transferred to the surface of the balloon 30.

Next, the inflation fluid is suctioned (i.e., sucked) and discharged from the inside of the balloon 30 through the proximal opening 27 of the hub 26. As a result, the balloon 30 is deflated and folded. As a result, the balloon 30 can be used for expansion of a stenotic part in a living body lumen, such as a blood vessel, as a drug eluting balloon.

As described above, the drug transfer device 10 according to the present embodiment is the drug transfer device 10 configured to transfer the drug to the surface of the balloon 30, and includes the heat-shrinkable tube 11 and the drug layer 12 provided on the inner surface of the heat-shrinkable tube 11.

When the drug transfer device 10 configured as described above is placed to cover the balloon 30 and heated, the heat-shrinkable tube 11 shrinks. As a result, the drug layer 12 is transferred to the surface of the balloon 30 by the shrinking heat-shrinkable tube 11, and an appropriate amount of the drug can be relatively quickly and rather easily provided on the surface of the balloon 30. The drug transfer device 10 can relatively quickly and rather easily provide the drug layer 12 on the balloon 30, and thus, can be also used in the state of covering the balloon 30 after use, for example, in a clinical field regardless of a place of use. Therefore, for example, the drug transfer device 10 can be applied to the balloon 30 removed from the living body after being used for pre-dilation of the stenotic part to obtain the balloon 30 for post-dilation having the drug layer 12. Therefore, when a balloon for pre-dilation and a balloon for post-dilation are required, the single balloon 30 can fulfill the two roles. Since the drug layer 12 can be relatively quickly and rather easily provided on the balloon 30, the drug transfer device 10 having an appropriate amount of a drug can be used by, for example, selecting from a plurality of drug transfer devices 10 having different types and amounts of drugs at the time of using the balloon 30.

In accordance with an aspect, the drug transfer device 10 further has the adhesive layer 13 provided on the inner surface of the drug layer 12. As a result, the adhesive layer 13 adheres to the surface of the balloon 30, and the drug layer 12 can be rather effectively transferred to the surface of the balloon 30.

In accordance with another aspect, the drug layer 12 is water-insoluble and the adhesive layer 13 is water-soluble. As a result, when the water-soluble adhesive layer 13 is formed on the inner surface of the water-insoluble drug layer 12, the dissolution of the drug in the drug layer 12 can be suppressed by the material of the adhesive layer 13 containing water. In addition, the balloon 30 contains moisture at the time of transferring the drug layer 12, and thus, favorable adhesiveness can be exhibited, and the dissolution of the drug in the drug layer 12 can be suppressed. Therefore, the surface of the balloon 30 may be wetted before the balloon 30 is covered with the drug transfer device 10. In addition, the balloon 30 removed from the living body after the pre-dilation is highly likely to contain moisture, and the adhesiveness can be improved.

In accordance with a further aspect, the water-insoluble drug in the drug layer 12 contains at least one selected from the group including rapamycin, paclitaxel, docetaxel, and everolimus. As a result, restenosis of the stenotic part in the blood vessel can be favorably suppressed by the drug layer 12.

In accordance with an aspect, the drug in the drug layer 12 may contain at least one selected from the group including a water-insoluble drug, a water-soluble drug, and a hydrophilic polymer. As a result, it is possible to apply, to the drug layer 12, a drug that is appropriate for conditions and the like, alone or in combination, with various other drugs.

The adhesive layer 13 may exert an adhesive force when heated. As a result, the adhesive layer 13 is also heated when the heat-shrinkable tube 11 is heated, and the adhesive layer 13 exhibits the adhesive force. Therefore, it is possible to prevent the adhesive layer 13 from adhering to an unintended position before the heating. Therefore, the heat-shrinkable tube 11 can be attached to an appropriate position on the surface of the balloon 30 after positioning the drug layer 12 with respect to the balloon 30 with high precision.

The heat-shrinkable tube 11 has the fragile portion 16 that is more fragile than other portions of the heat-shrinkable tube 11. As a result, the heat-shrinkable tube 11 that has been heat-shrunk can be rather easily removed from the surface of the balloon 30 by breaking the fragile portion 16.

In addition, the present invention also can include a method for forming a drug layer configured to provide a drug on the surface of the balloon 30. The method for forming a drug layer includes covering the balloon 30 with the drug transfer device 10 in which the inner surface of the heat-shrinkable tube 11 is coated with the drug layer 12; transferring the drug layer 12 to the surface of the balloon 30 by heating the heat-shrinkable tube 11 to shrink; and removing the heat-shrinkable tube 11 from the drug layer 12.

In the method for forming a drug layer configured as described above, the drug layer 12 can be transferred to the surface of the balloon 30 by the heat-shrinkable tube 11 that shrinks as the balloon 30 is covered with the drug transfer device 10 and heated. As a result, the drug layer 12 is transferred to the surface of the balloon 30 by a shrinking force of the heat-shrinkable tube 11, and an appropriate amount of the drug can be relatively quickly and rather easily provided on the surface of the balloon 30.

In the transferring of the drug layer 12 to the surface of the balloon 30 by heating the heat-shrinkable tube 11 to shrink, the adhesive layer 13 provided on the inner surface of the drug layer 12 may be attached to the surface of the balloon 30. As a result, the adhesive layer 13 adheres to the surface of the balloon 30, and the drug layer 12 can be rather effectively transferred to the surface of the balloon 30.

In the covering of the balloon 30 with the drug transfer device 10, the balloon 30 removed from the living body may be covered with the drug transfer device 10. As a result, the balloon 30 used for pre-dilation of a target portion of the living body can be removed, and then, the drug layer 12 can be provided on the same balloon 30 for reuse in post-dilation.

Note that the present disclosure is not limited to only the above-described embodiment, and various modifications can be made by those skilled in the art within a technical idea of the present invention. For example, the balloon catheter 50 can be a rapid exchange type, but may be an over-the-wire type.

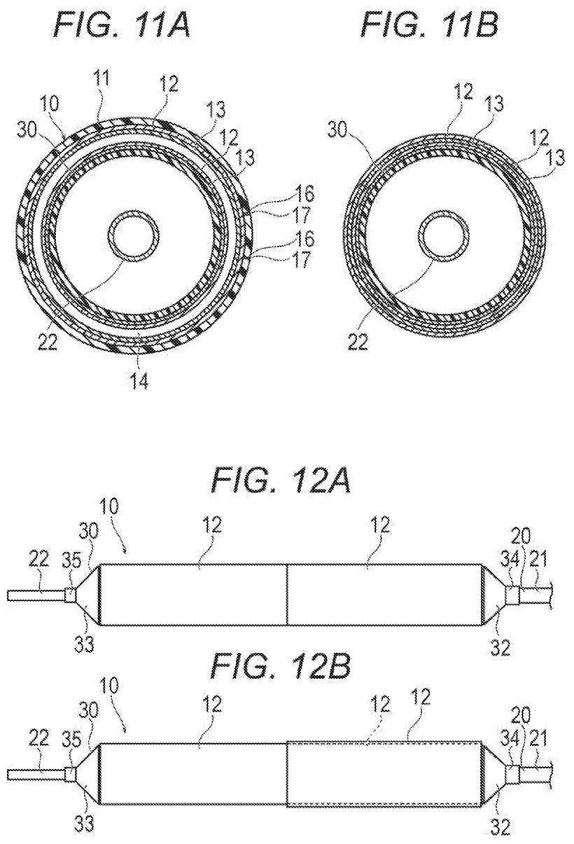

In addition, the drug layer 12 may be provided so as to overlap with one balloon 30 by a plurality of drug transfer devices 10. For example, the drug layer 12 can be attached to the balloon 30 by the adhesive layer 13 using the drug transfer device 10 as illustrated in FIG. 10, and then, the balloon 30 can be inserted into the through-hole 14 of the other drug transfer device 10 as illustrated in FIG. 11A. Thereafter, the balloon 30 is inflated to reduce the diameter of the heat-shrinkable tube 11 in the same manner as the above-described method. Then, the heat-shrinkable tube 11 is peeled off from the balloon 30, and the two drug layers 12 can be provided so as to overlap each other for the medical instrument as illustrated in FIG. 11B. Three or more drug layers 12 may be provided for the medical instrument by additionally using the drug transfer device 10. It is possible to adjust a loading amount of a drug by forming two or more drug layers 12 so as to overlap each other for the medical instrument.

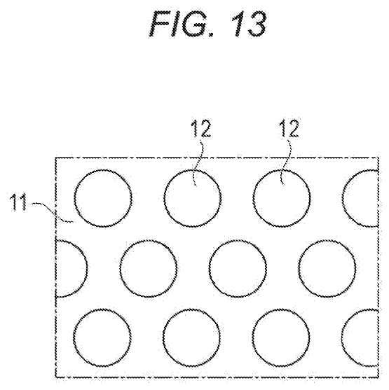

In addition, at least two drug layers 12 may be formed in a region adjacent to the balloon 30 so as not to overlap each other as illustrated in FIG. 12A. In accordance with an embodiment, the at least two drug layers 12 are transferred to the balloon 30 by different drug transfer devices 10. Note that the different drug layers 12 may partially overlap each other. In addition, when a plurality of drug transfer devices 10 are used, a plurality of drug layers 12 can be provided side by side while overlapping each other for one medical instrument as illustrated in FIG. 12B. Therefore, the drug layers 12 can be provided to overlap each other, provided side by side, or provided side by side while overlapping each other, for one medical instrument (for example, the balloon 30) by using the plurality of drug transfer devices 10.

In addition, when a plurality of drug transfer devices 10 having different types of loaded drugs are used, the types of drugs in the plurality of drug layers 12 can be made different as illustrated in FIGS. 11A to 12B. For example, a plurality of types of drugs such as a drug for suppressing restenosis, an antiplatelet drug, and a blood anticoagulant can be loaded in combination.

In addition, a target to which the drug is transferred by the drug transfer device 10 is not limited to the balloon 30 as long as the target is a medical instrument that is used by being inserted into a living body, and may be, for example, a stent, a covered stent, an implant, or the like.

In addition, the balloon 30 is folded in the deflated state in the balloon catheter 50, but is not necessarily folded. That is, the balloon 30 may be formed of a material having elasticity to be inflated while the thickness of the balloon 30 can be reduced.

In addition, the adhesive layer 13 is not necessarily provided as long as the drug layer 12 can be transferred to the surface of the balloon 30. In addition, the additive contained in the drug layer 12 may function as an adhesive for attachment to the balloon 30.

In addition, a protective film that can be peeled off from the adhesive layer 13 may be attached to the inner surface of the adhesive layer 13. As a result, it is possible to suppress, for example, dust and the like from adhering to the adhesive layer 13 before use. The protective film can be rather easily peeled off before the adhesive layer 13 is transferred to the balloon 30. A thickness of the protective film is not particularly limited, but can be 0.005 mm to 0.05 mm, for example.

Further, the drug layer 12 may be partially provided on the inner surface of the heat-shrinkable tube 11 as in a modification illustrated in FIG. 13. Note that a shape of the range where the drug layer 12 is provided is not particularly limited. Therefore, the drug transfer device 10 can arbitrarily set the range where the drug layer 12 is provided.

The detailed description above describes embodiments of a drug transfer device configured to transfer a drug to a surface of a medical instrument such as a balloon, and a method for forming a drug layer on a surface of a medical instrument. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A drug transfer device configured to transfer a drug to a surface of a medical instrument used by being inserted into a living body, the drug transfer device comprising:
a heat-shrinkable tube; and
a drug layer provided on an inner surface of the heat-shrinkable tube.

2. The drug transfer device according to claim 1, further comprising:
an adhesive layer provided on an inner surface of the drug layer.

3. The drug transfer device according to claim 1, wherein the drug layer is water-insoluble, and the adhesive layer is water-soluble.

4. The drug transfer device according to claim 1, wherein the drug in the drug layer contains at least one selected from a group including a water-insoluble drug, a water-soluble drug, and a hydrophilic polymer.

5. The drug transfer device according to claim 1, wherein the adhesive layer exhibits an adhesive force when heated.

6. The drug transfer device according to claim 1, wherein the heat-shrinkable tube has a fragile portion that is more fragile than other portions of the heat-shrinkable tube.

7. The drug transfer device according to claim 1, wherein the fragile portion of the heat-shrinkable tube is a series of small holes in the heat-shrinkable tube.

8. The drug transfer device according to claim 1, wherein the medical instrument is a balloon that is capable of inflating and deflating.

9. A method for forming a drug layer which provides a drug on a surface of a medical instrument configured to be inserted into a living body, the method comprising:
covering the medical instrument with a drug transfer device in which an inner surface of a heat-shrinkable tube is coated with the drug layer;
transferring the drug layer to the surface of the medical instrument by heating the heat-shrinkable tube to shrink; and
removing the heat-shrinkable tube from the drug layer on the surface of the medical instrument.

10. The method for forming a drug layer according to claim 9, further comprising:
attaching an inner surface of the drug layer to the surface of the medical instrument with an adhesive.

11. The method for forming a drug layer according to claim 9, wherein the covering of the medical instrument with the drug transfer device, the method comprising:
covering the medical instrument removed from an inside of the living body with the drug transfer device.

12. The method for forming a drug layer according to claim 9, wherein the medical instrument is a balloon, a guidewire, a guiding sheath, a guiding catheter, or a stent which is capable of inflating and deflating.

13. A method for forming a drug layer which provides a drug on a surface of a balloon configured to be inserted into a living body, the method comprising:
injecting a predetermined amount of an inflation fluid into the balloon to inflate the balloon;
inserting the inflated balloon into a through-hole of a heat-shrinkable tube, the heat-shrinkable tube having an inner surface coated with a drug layer;
transferring the drug layer to the surface of the inflated balloon by heating the heat-shrinkable tube; and
removing the heat-shrinkable tube from the drug layer on the surface of the balloon.

14. The method for forming a drug layer according to claim 13, further comprising:
further inflating the balloon after the balloon has been inserted into the through-hole of the heat-shrinkable tube.

15. The method for forming a drug layer according to claim 13, wherein in the transferring of the drug layer to the surface of the balloon, the method comprising:
attaching an adhesive layer provided on an inner surface of the drug layer to the surface of the balloon.

16. The method for forming a drug layer according to claim 15, further comprising:
removing the inflation fluid from an inside of the balloon after shrinking the heat-shrinkable tube onto the surface of the balloon.

17. The method for forming a drug layer according to claim 13, wherein the balloon includes a straight portion formed at a center in the axial direction, a proximal tapered portion located on a proximal side of the straight portion, and a distal tapered portion located on the distal side of the straight portion, the method comprising:
attaching the heat-shrinkable tube to only the straight portion of the balloon.
